(19) United States
(12) Patent Application Publication
(10) Pub. No.: US 2017/0135874 A1
(43) Pub. Date: May 18, 2017

(54) MULTILAYER WRAPPER FILM FOR HYGIENE PRODUCT

(71) Applicant: MONDI Consumer Packaging Technologies GmbH, Gronau (DE)

(72) Inventors: Herbert BADER, Nordwalde (DE); Timothy STOLTZ, West Chester, OH (US); Ralf NIEPELT, Gronau (DE)

(21) Appl. No.: 14/941,761

(22) Filed: Nov. 16, 2015

(51) Int. Cl. A61F 13/551; B32B 7/12; B32B 27/08; B32B 27/30; B32B 27/32; A61F 13/56; B32B 3/26

(52) U.S. Cl. CPC A61F 13/5514; A61F 13/5611; B32B 7/12; B32B 3/266; B32B 27/302; B32B 27/32; B32B 27/08; B32B 2555/00; B32B 2439/80

(57) ABSTRACT

A multilayer wrapper film for a hygiene product has a first outer layer, a second outer layer, and a pressure-sensitive adhesive layer between the first outer layer and the second outer layer and forming therewith respective adhesive bonds. The adhesive bond between the pressure-sensitive adhesive layer and the first outer layer is stronger than the adhesive bond between the pressure-sensitive adhesive layer and the second outer layer. An overall thickness of the first and second outer layers with the pressure-sensitive adhesive layer arranged therebetween is between 20 μm and 80 μm.

### FIELD OF THE INVENTION

The present invention relates to a multilayer wrapper film. More particularly this invention concerns such a film used to wrap a hygiene product such as a sanitary napkin, panty liner incontinence product, and other flat, absorbent hygiene products that are packaged individually in a wrapper film.

### BACKGROUND OF THE INVENTION

A standard sanitary-napkin wrapper film extends beyond the sanitary napkin in a flattened-out state. During production, the ends are then folded over toward one another along fold lines extending transversely of the longitudinally elongated napkin, producing a three-layered configuration in which each of the ends of the sanitary napkin is folded onto a center zone of the sanitary napkin and is protected by the wrapper film. The wrapper can be fastened by lateral embossing and welding and seals and/or by an adhesive strip. Such wrapper films and sanitary napkins individually wrapped inside said wrappers are known from WO 1991/018,574 and U.S. Pat. No. 5,181,610.

The individual sanitary napkins are advantageously positioned extending transverse to a production direction on an upper face of a strip extending in the production direction of the wrapper film. Then the edges of the wrapper film with the sanitary napkins positioned thereon are folded over to achieve the known C-shaped folded form of the individually wrapped sanitary napkins. The wrapper film is then fastened both by embossing and by applying an adhesive strip along lines extending transversely of the production direction between the successive sanitary napkins. Finally, the sleeve formed in this manner is cut along the centers of the embossed lines into sections each containing one sanitary napkin. The result is a very inexpensive yet closed single-use package that can be easily opened but that keeps the product clean, even if being carried in a cluttered handbag or a pocket.

These wrapper films have an overall thickness of only 16 μm and are coextruded in three layers with a silicon coating on the inner face touching the sanitary napkin. Known wrapper films are also ordinarily colored, for which purpose a white batch or another colored batch may be provided. Furthermore, the films may also be imprinted, embossed or provided with notches.

A user may view a very thin film as being cheap, although this impression can be countered to a certain extent by embossing. This problem is of particular significance because, in the interest of conserving materials, even thinner film thicknesses of 13 μm to 14 μm for example are desirable.

To produce the individually wrapped sanitary napkins, a web of the wrapper film is supplied and is provided on the side with the silicon coating with a pressure-sensitive hot-melt adhesive applied at a temperature of 160° C. to 180° C., for example. In very thin films, even this amount of thermal stress can result in undesirable deformation.

The pressure-sensitive adhesive is applied in limited regions, and the individual sanitary napkins are applied to the pressure-sensitive adhesive. The silicon coating ensures that the pressure-sensitive adhesive will subsequently remain on the underside of the sanitary napkin and can be reliably separated from the wrapper film.

In addition to the disadvantages already mentioned, it is important to consider that a risk of tearing exists, both during production and later during use, due to the low overall thickness of the wrapper film.

Furthermore, applying the pressure-sensitive adhesive in a precisely predefined pattern and then arranging the individual sanitary napkins in the correct position thereon requires careful processing and high instrumentation expenditure.

### OBJECTS OF THE INVENTION

It is therefore an object of the present invention to provide an improved multilayer wrapper film for a hygiene product.

Another object is the provision of such an improved multilayer wrapper film for a hygiene product that overcomes the above-given disadvantages, in particular that is made without having to apply pressure-sensitive adhesive in the molten state directly during the production of the individually wrapped sanitary napkins.

Since the wrapper film is supplied as an intermediate product that is then further processed, another object of the invention is to facilitate such further processing.

### SUMMARY OF THE INVENTION

A multilayer wrapper film for a hygiene product has according to the invention a first outer layer, a second outer layer, and a pressure-sensitive adhesive layer between the first outer layer and the second outer layer and forming therewith respective adhesive bonds. The adhesive bond between the pressure-sensitive adhesive layer and the first outer layer is stronger than the adhesive bond between the pressure-sensitive adhesive layer and the second outer layer. An overall thickness of the first and second outer layers with the pressure-sensitive adhesive layer arranged therebetween is between 20 μm and 80 μm.

According to the invention, the pressure-sensitive adhesive, which will later be used for positioning the sanitary napkin in the crotch area of a pair of underwear during use, is initially in the multilayer wrapper film, and secure bonding of the first outer layer to the at least one pad of the sanitary napkin causes the multilayer wrapper film to tear open in localized areas, leaving the first outer layer with the pressure-sensitive adhesive layer on the backing of the sanitary napkin.

The subject matter of the invention therefore also comprises an individually wrapped sanitary napkin where the multilayer wrapper film extends beyond the sanitary napkin, and in which the at least one pad of the sanitary napkin is non-releasably bonded to the first outer layer. Non-releasable within the context of the present invention is understood to mean that the bond between the sanitary napkin or the at least one pad of the sanitary napkin cannot separate from the first outer layer when the sanitary napkin is pulled away from the wrapper film, and instead, the first outer layer tears along the edge of the nonrelease bond with the pressure-sensitive adhesive beneath it, and is then separated along a planar area from the second outer layer.

Various chemically and/or physically curing adhesives may be provided for producing the nonrelease bond. In addition to solvent-containing adhesives and multicomponent adhesives, in principle hot-melt adhesives of sufficient strength may also be considered.

In the zones of the first outer layer to be non-releasably bonded to the sanitary napkin or to a pad of the sanitary napkin, the adhesive may be applied over a continuous flat area or in a pattern. If adhesive is applied in a pattern, it may also be applied around the full perimeter, for example, to ensure a secure hold at those points that are subject to the greatest stress.

A number of advantages result within the scope of the invention. First, a greater overall thickness of the wrapper film of between 20 μm and 80 μm is acceptable, even based on cost, because the pressure-sensitive adhesive for the sanitary napkin is already integrated into the wrapper film as an intermediate product. The overall thickness of between 20 μm and 80 μm results in an impression of higher quality from the point of view of a consumer.

Furthermore, within the scope of the invention, a silicon coating or another release coating on the exterior of the multilayer wrapper film can be dispensed with entirely, so that the wrapper film can also be easily thermally welded with a suitable selection of materials. For example, once it has been folded into a C-shape, it can be fastened by thermal-weld points or seams, allowing the application of a separate adhesive strip as a closure to be dispensed with.

A further advantage is that the application of a simple, nonrelease adhesive can be carried out more easily than coating with a molten pressure-sensitive adhesive.

Within the scope of the invention, it must be ensured that the adhesive bond between the pressure-sensitive adhesive layer and the first outer layer is stronger than the adhesive bond between the pressure-sensitive adhesive layer and the second outer layer, so that, when the sanitary napkin is pulled away from the wrapper film, the pressure-sensitive adhesive will separate from the second outer layer and will remain together with the corresponding portion of the first outer layer on the backing of the sanitary napkin. To this end, the second outer layer can have a release sublayer on which lies the pressure-sensitive adhesive layer. Additionally or alternatively, a matching layer of adhesion promoter can be provided between the first outer layer and the pressure-sensitive adhesive layer to increase the strength of the adhesive bond.

According to the invention, the release sublayer may contain or consist of a siloxane, especially a silicone, preferably a silicone elastomer or silicone rubber. Between the release sublayer and the second outer layer there may be an adhesion agent sublayer.

In principle, the first outer layer and the second outer layer can each be formed from only one film layer or from a plurality of film layers.

According to the invention, a multilayer wrapper film is provided in which the pressure-sensitive adhesive layer is protected between the two outer layers. The wrapper film can be produced in various ways.

According to a first variant, the two outer layers and the pressure-sensitive adhesive layer are coextruded together, resulting in a particularly simple process. In this case, a subsequent joining of various layers or application of a coating is not necessary, in contrast to the prior art.

According to a preferred embodiment, the coextruded film that is glued or welded to the pad has the following layers:
a first outer layer 10 to 15 μm thick of PE-LD, PE-LLE, and/or EVA,
a pressure sensitive adhesive 15 to 20 g/m² of SBS, UV-hardenable acrylic and/or SIS,
a release sublayer 2.5 to 5 μm thick of a Si compound (in particular a silicon elastomer),
a tie layer 2.5 to 5 μm thick, and
a second outer layer 10 to 15 μm thick of PE-LD, PE-LLD, and/or EVA
   for a total thickness of 40 to 65 μm.

Alternatively, the pressure-sensitive adhesive layer can also be made of a thermoplastic plastic, in which case the first outer layer and the second outer layer are produced separately and are laminated using the pressure-sensitive adhesive layer as a hot-melt adhesive. The pressure-sensitive adhesive layer then fulfills two different purposes. During manufacture, the pressure-sensitive adhesive layer in its molten state serves to join the first outer layer to the second outer layer in a laminating process; however, the pressure-sensitive adhesive layer nevertheless permanently retains its adhesive characteristics and enables the sanitary napkin to be fastened in the crotch area of a pair of underwear in the described manner during use.

According to another preferred of this invention, the film laminate that is glued or welded to the pad can have the following layers:
first film, 10 to 15 μm thick of PE-LD, PE-LLD, and or EVA,
pressure-sensitive adhesive 15 to 20 g/m² of SBS, a UV-hardenable acrylic, and/or SIS, and
second conventional wrapper film with a 15 μm silicone coating, the film being made of PE-LE, PE-LLD, PP, and or PE-HD+silicone
   for a total thickness of 40 to 50 μm.

The pressure-sensitive adhesive layer can be provided over the entire contact area between the first outer layer and the second outer layer. It is also possible, however, for the pressure-sensitive adhesive to be applied only in a pattern or in sections, with such a partial-surface application being particularly appropriate for a laminating process. A uniform pattern of points or a grid can be provided for the purpose of conserving pressure-sensitive adhesive overall; in addition, with a uniform pattern, when the sanitary napkin is applied to the wrapper film, it is not necessary to maintain a predefined pattern repeat, that is, a precise positioning. In principle, however, it is also possible to provide the pressure-sensitive adhesive only in sections that then later form the fastening regions for the sanitary napkin.

If a partial-surface arrangement is provided within the framework of a coextrusion process, the interstices may optionally be filled in with a non-adhesive material.

The pressure-sensitive adhesive can be formed, for example, with a styrene block copolymer base, in particular a styrene-isoprene block copolymer (SIS) base. Styrene block copolymers are particularly suitable for coextrusion of the entire multilayer wrapper film.

Acrylate adhesives are particularly suitable for lamination using the pressure-sensitive adhesive as a hot-melt adhesive.

Within the scope of the invention, the adhesive bond between the individual layers is an important factor. However, it must also be ensured that the first outer layer of the multilayer wrapper film will tear open along the edges of the permanent bond between the sanitary napkin or the pad of the sanitary napkin and the first outer layer, that is along the outer periphery of the napkin. To this end, the first outer layer may be made relatively fragile and brittle by a suitable selection of the polymer component and/or by adding auxiliary substances, allowing it to tear open easily.

Additionally or alternatively, the first outer layer can also have along the edges of the non-releasably bonded surface a tear line formed, for example, by a laser or by punching. Within the scope of such an embodiment, however, the sanitary napkin or a pad of the sanitary napkin must then be arranged in the proper position.

To enable low production costs and good functional characteristics, the first outer layer and the second outer layer according to a preferred embodiment of the invention each comprise a polyolefin, in particular polyethylene (PE), as the main polymeric constituent. Within the scope of such an embodiment, once the wrapper film has been folded, it can be sealed or fastened by thermal welding.

The thickness of the first outer layer and the second outer layer is typically between 5 μm and 30 μm, and the thickness of the pressure-sensitive adhesive layer is typically between 5 μm and 25 μm.

Once the sanitary napkin has been pulled away from the wrapper film, the pressure-sensitive adhesive layer is fastened across the first outer layer on the sanitary napkin or on the pad of the sanitary napkin. In this case, the first outer layer forms a functional layer of the sanitary napkin as a whole, for example a rear surface of the sanitary napkin.

Within the scope of such an embodiment, only one pad of the sanitary napkin is provided and is applied to the first outer layer, and preferably bonded over the entire surface area thereof. The first outer layer then forms a rear surface of the sanitary napkin once the sanitary napkin has been pulled away from the wrapper film. This rear surface can also have pores to provide breathability properties.

To produce breathable pores in the first outer layer, at least the first outer layer can be stretched. In this case, the first outer layer can contain filler materials, with stretching resulting in the formation of breathable pores where the particles of filler material are located. If the multilayer wrapper film as a whole is produced by laminating the first outer layer to the second outer layer, stretching of only the first outer layer prior to lamination is sufficient.

However, if the multilayer wrapper film is produced as an extrusion film, the entire coextrusion film must be stretched in the described manner in order to form breathable pores where the particles of filler material are located in the first outer layer.

In principle, filler materials may also be provided for coloring the wrapper film and/or for optimizing tearing behavior, particularly the tearing of the first outer layer away from the nonrelease bond with the sanitary napkin or a pad of the sanitary napkin.

According to an alternative embodiment of the invention, the entire rear surface of the sanitary napkin is covered by the pressure-sensitive adhesive layer. Within the scope of such an embodiment, the rear surface of the pad of the sanitary napkin is non-releasably bonded to the outer layer, with the non-releasably bonded area amounting to 20% to 80% of the area of the sanitary napkin in its flattened-out state.

In particular, a plurality of sections can also be non-releasably bonded in the described manner. Particularly preferably, the rear surface and the first outer layer are inseparably bonded along two strips that preferably extend parallel to one another longitudinally of the sanitary napkin. The longitudinal direction of the sanitary napkin corresponds to the extension of a crotch area of a pair of underpants.

### BRIEF DESCRIPTION OF THE DRAWING

The above and other objects, features, and advantages will become more readily apparent from the following description, reference being made to the accompanying drawing in which:

FIG. 1 is a top view of a flattened sanitary napkin on a section of a wrapper film during a first production step;

FIG. 2 is a perspective view of the sanitary napkin wrapped individually in the wrapper film;

FIG. 3a is a large-scale longitudinal section along line IIIa-IIIa of FIG. 1;

FIG. 3b is a view like FIG. 3a, but showing the assembly after the package has been unfolded and the sanitary napkin has been pulled away from the wrapper film;

FIG. 4a shows the arrangement as to FIG. 3a in an alternative embodiment; and

FIG. 4b shows the arrangement of FIG. 4a with the sanitary napkin pulled away from the wrapper film as in FIG. 3b.

### SPECIFIC DESCRIPTION OF THE INVENTION

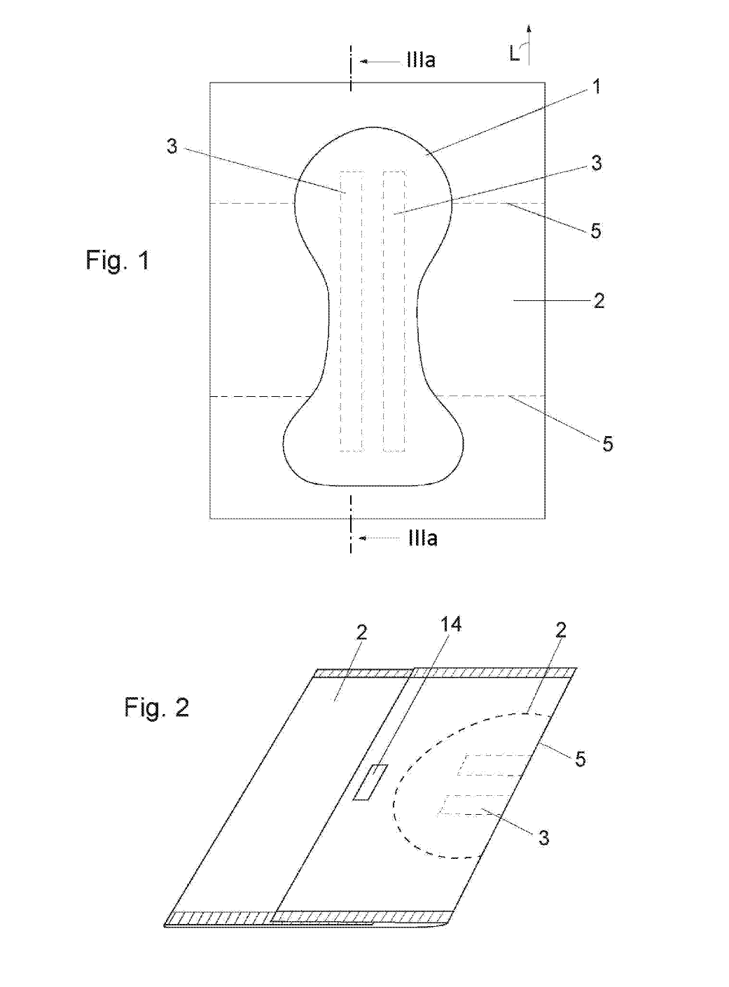

As seen in FIG. 1 a dumbbell-shaped sanitary napkin 1 in a flattened-out state lies atop a rectangular section of wrapper film 2 that actually is part of a long strip of the film extending in a production direction lying in the view plane and perpendicular to a longitudinal direction L of the napkin 1.

To allow a sanitary napkin 1 to be fastened in the crotch area of a pair of underpants, the underside of the sanitary napkin is provided as shown in FIG. 1 with a pair of adhesive regions 3 containing a pressure-sensitive adhesive 4. The regions 3 are parallel strips parallel to one another along the longitudinal direction L of the napkin 1.

The napkin 1 can also be provided in a central zone thereof with unillustrated lateral wings that are first folded over toward the top and that also have adhesive regions. Within the scope of such an unillustrated embodiment a separate cover is then required for the wings that are folded over toward the top.

FIG. 2 shows the napkin 1 packaged individually with a wrapper film 2 and made by folding the flattened arrangement of FIG. 1 together along fold lines 5 extending transverse to the longitudinal direction L into a C-shape, so that the longitudinally opposite ends of the napkin 1 are in a central zone defined between the fold lines 5.

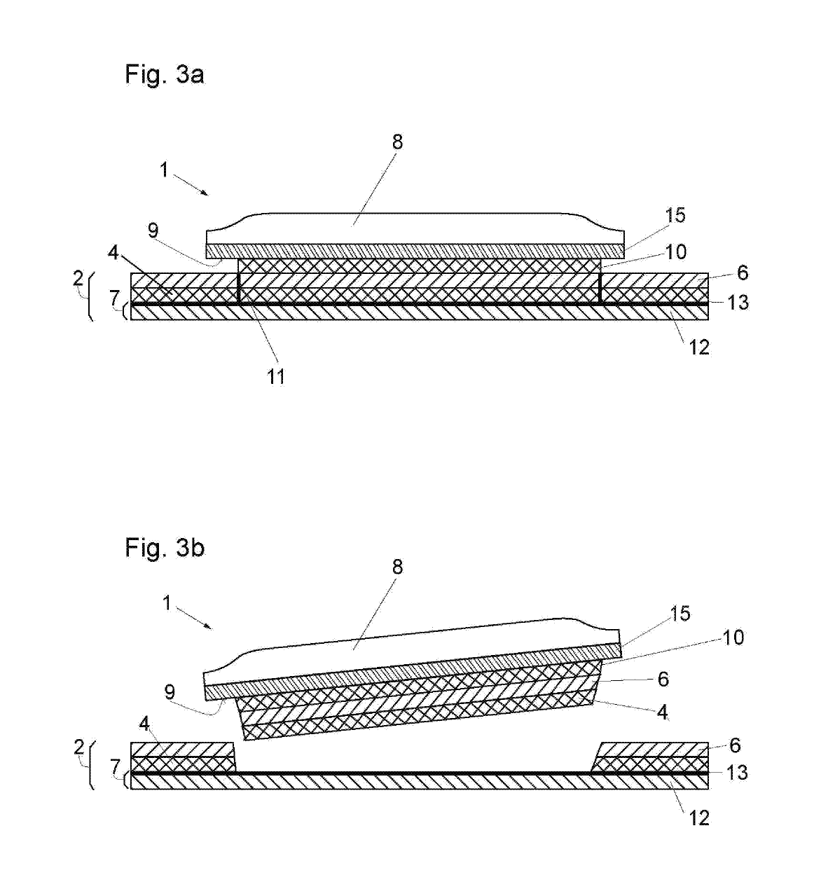

According to FIG. 3a, the wrapper film 2 has several layers and comprises a first or upper outer layer 6, a second or lower outer layer 7, and the layer 4 of pressure-sensitive adhesive between the first outer layer 6 and second outer layer 7. All the layers 4, 6, and 7 are in surface contact with each other with no gaps or voids.

The napkin 1 on the wrapper film 2 comprises a pad 8 itself formed by a plurality of nonwoven layers under an unillustrated fluid-permeable top cover layer. The napkin 1 further has a backing sublayer 15 that preferably has breathable properties but is watertight.

The napkin 1 is sealed to the first outer layer 6 by a nonrelease adhesive layer 10 forming a rear surface 9 of the back sublayer 15 of the napkin 1. The nonrelease adhesive layer 10 is applied, according to the embodiment of FIG. 3a, within a tear line 11 has already been formed in the first outer layer 6 by punching or by a laser and that has dumbbell shape conforming to that of the outer periphery of the napkin 1. This line is optional. Alternatively, the first outer layer may also be designed to easily tear away from the rest of the wrapper film 2 as the napkin 1 is pulled away.

According to the invention, the adhesive bond of the adhesive layer 4 to the first outer layer 6 is stronger than the adhesive bond between the adhesive layer 4 and the second outer layer 7. Thus when the pad 8 is pulled up, it will take the layers 10, 6, and 4 with them and leave the layer 7 behind. as shown in FIG. 3b.

In the embodiment shown, the second outer layer has two sublayers, namely a base sublayer 12 and, bordering the adhesive layer 4, a release sublayer 13 that has a particularly weak adhesive bond relative to the adhesive layer 4 and that ensures separation between the layers 4 and 7. In the embodiment shown, the first outer layer 6 is formed by only one film layer; however, a multilayer embodiment is also possible.

In the embodiment shown, the thickness of the base sublayer 12 and the first outer layer 6 is within a range of 5 μm and 20 μm, for example approximately 13 μm, and it is made of a polyolefin, preferably polyethylene (PE), so that in the folded state according to FIG. 2, the first outer layer 6 can be bonded to the base sublayer 12 of the second outer layer 7 by a thermal weld 14, allowing a separate adhesive strip for sealing the package according to FIG. 2 to be dispensed with.

When the napkin 1 is pulled away from the wrapper film 2, according to FIG. 3b, the first outer layer 6 with the adhesive layer 4 remains on the napkin 1 along the nonrelease adhesive layer 10, thereby producing the adhesive regions 3 shown in FIG. 1.

The adhesive regions 3 formed in this manner preferably extend across between 20% and 80% of the area of the napkin 1 in its flattened-out state (see FIG. 1).

The wrapper film 2 can be produced entirely by coextrusion or also by lamination. For example, the first outer layer 6 and the second outer layer 7 can be laminated together by the adhesive layer 4 if it is thermoplastic and is introduced in the molten state between the first outer layer 6 and the second outer layer 7. The adhesive layer 4 then serves during lamination as a hot-melt adhesive.

The adhesive layer 4 can be formed, for example, of a styrene-block copolymer base, in particular SIS, or with an acrylate base.

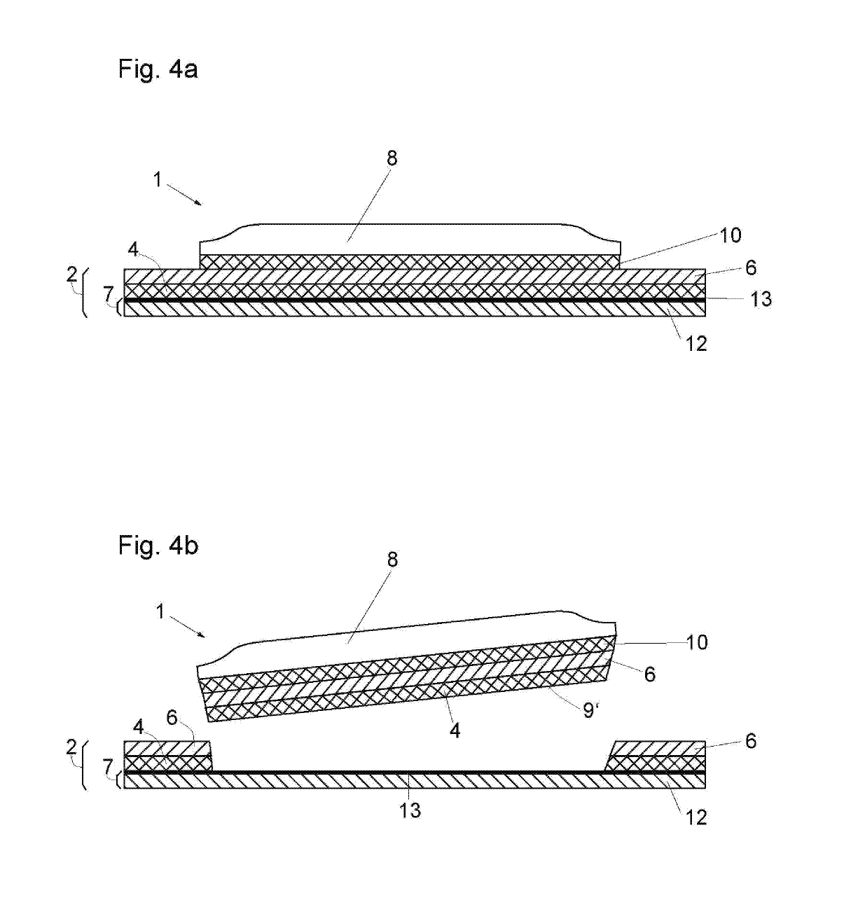

FIGS. 4a and 4b show an alternative embodiment of the invention, in which only the pad 8 of the napkin 1 is bonded by the nonrelease adhesive layer 10 to the first outer layer 6.

The pad 8 is preferably bonded across its entire surface area to the nonrelease adhesive layer 10, and later, when the napkin 1 is pulled away from the wrapper film 2, the first outer layer 6 remains attached to of the pad 8 by the nonrelease adhesive layer 10 and the adhesive layer 4 a rear surface 9′ of the napkin 1, thereby enabling further simplification of the design.

To achieve breathability in an embodiment according to FIGS. 4a and 4b, the first outer layer 6 can be provided with filler materials, for example chalk, during its production, whereupon the first outer layer 6 is then stretched, forming breathable pores where the particles of filler material are located.

If the first outer layer 6 is laminated to the second outer layer 7, it is sufficient merely to stretch the first outer layer 6 prior to lamination. If, in contrast, the wrapper film 2 as a whole is produced by coextrusion, the entire coextrusion film must be subjected to a stretching process in order to provide the first outer layer 6 with breathable properties as described.

We claim:

1. A multilayer wrapper film for a hygiene product, the film comprising:
a first or upper outer layer;
a second or lower outer layer; and
a pressure-sensitive adhesive layer between the first outer layer and the second outer layer and forming therewith respective adhesive bonds, the adhesive bond between the pressure-sensitive adhesive layer and the first outer layer being stronger than the adhesive bond between the pressure-sensitive adhesive layer and the second outer layer, an overall thickness of the first and second outer layers with the pressure-sensitive adhesive layer arranged therebetween being between 20 μm and 80 μm.

2. The multilayer wrapper film defined in claim 1, wherein the second outer layer has a release sublayer that lies against the pressure-sensitive adhesive layer.

3. The multilayer wrapper film defined in claim 1, wherein a thickness of the first outer layer and the second outer layer together is between 5 μm and 30 μm and a thickness of the pressure-sensitive adhesive layer is between 5 μm and 25 μm.

4. The multilayer wrapper film defined in claim 1, wherein the first outer layer, the second outer layer and the pressure-sensitive adhesive layer are coextruded.

5. The multilayer wrapper film defined in claim 1, wherein the pressure-sensitive adhesive layer is formed of a styrene block copolymer base.

6. The multilayer wrapper film defined in claim 5 wherein the copolymer base is a styrene-isoprene-styrene block copolymer base.

7. The multilayer wrapper film defined in claim 1, wherein the pressure-sensitive adhesive layer is thermoplastic and the first outer layer and the second outer layer are produced separately and are laminated using the pressure-sensitive adhesive layer as a hot-melt adhesive.

8. The multilayer wrapper film defined in claim 7, wherein the pressure-sensitive adhesive layer has an acrylate base.

9. The multilayer wrapper film defined in claim 1, wherein the first outer layer and the second outer layer each have a polyolefin as a polymeric main constituent.

10. The multilayer wrapper film defined in claim 9, wherein the polyolefin is polyethylene.

11. The multilayer wrapper film defined in claim 1, wherein at least the first outer layer is stretched.

12. The multilayer wrapper film defined in claim 11, wherein the first outer layer contains filler materials and has breathable pores formed by stretching.

13. The multilayer wrapper film defined in claim 1, further comprising:
a sanitary pad having an outer periphery; and
a nonrelease bond between a rear face of the pad and an outer face of the first outer layer.

14. The multilayer wrapper film defined in claim 13, wherein the pad bonded over its entire surface area within the periphery to the first outer layer so that the first outer layer forms a rear surface of the sanitary napkin.

15. The multilayer wrapper film defined in claim 13, wherein a rear surface over between 20% and 80% of a surface area of the sanitary napkin within its periphery in a flattened state.

16. The multilayer wrapper film defined in claim 13, wherein the rear face of the pad and the outer face of the first layer are secured together by the nonrelease bond along two strips.

17. The multilayer wrapper film defined in claim 13, wherein the first outer layer has a tear line at an outer periphery of the nonrelease bond.
